(19) United States
(12) Patent Application Publication
(10) Pub. No.: US 2012/0046582 A1
(43) Pub. Date: Feb. 23, 2012

(54) CHEST SEAL BANDAGE AND OTHER MEDICAL DEVICES FOR NIGHT USE

(75) Inventors: Lance David Hopman, Tigard, OR (US); Lane Michael Johnson, Tualatin, OR (US); Jaret Dwayne Schaffer, Lake Oswego, OR (US); Samuel Scheinberg, Otis, OR (US)

(73) Assignee: The Seaberg Company, Inc.

(21) Appl. No.: 12/965,667

(22) Filed: Dec. 10, 2010

Related U.S. Application Data
(60) Provisional application No. 61/401,643, filed on Aug. 17, 2010.

(51) Int. Cl. A61F 5/00; A61B 19/04; A61F 5/04; A61F 13/02; A61B 17/00

(52) U.S. Cl. 602/4; 128/887; 602/54; 606/203; 602/5; 2/161.7

(57) ABSTRACT

A medical dressing for chest wounds, including a one-way valve that can be covered so as to protect against intrusion of airborne contaminants and to prevent passage of fluids in either direction, and including indicia visible using night vision devices but not visible in visible light to the naked eye in some embodiments. The indicia visible using night vision devices may be included in other medical apparatus, as well, so that the medical dressings or other apparatus can be used effectively during darkness, in combat situations, without being seen by enemy combatants not equipped with night vision devices.

### CROSS-REFERENCE TO RELATED APPLICATIONS

This application claims the benefit of the filing date of U.S. Provisional Patent Application Ser. No. 61/401,643, filed Aug. 17, 2010.

### BACKGROUND OF THE INVENTION

The present invention relates to medical devices such as a medical bandage or dressing for treating open chest wounds and other injuries, and to use of such medical devices in darkness.

An open pneumothorax occurs when air is able to enter the chest cavity surrounding the lungs through an open chest wound, such as may be sustained as a result of being stabbed or shot. The admission of air into the chest cavity through a wound in this manner inhibits the normal breathing process and so can be life threatening.

Standard treatment for an open pneumothorax is to cover the wound with an occlusive dressing which is taped to the patient's skin along all sides so as to seal the wound and prevent air from entering the chest cavity through the wound. If the wound is effectively sealed then normal breathing can be restored. However, if the seal is not perfect, more air may be drawn into and trapped in the chest cavity, causing an increase in pressure inside the chest cavity. This is known as a tension pneumothorax and is a potentially more serious condition which can quickly lead to respiratory and heart failure.

Tension pneumothorax can also occur if the patient has suffered a punctured lung, as air can escape from the lung through the puncture into the chest cavity each time the patient breathes. If the open chest wound has been sealed, the air entering the chest cavity through the lung puncture is trapped and the pressure inside the chest cavity rises, forcing the lung to collapse. Once the lung has collapsed, the pressure in the chest cavity will increase further so that the collapsed lung is pressed against the heart and the other lung. The blood flow from the heart to the lungs may be stopped if the pressure in the chest cavity increases above normal blood pressure.

Treatment of a tension pneumothorax with an open wound typically comprises using an occlusive dressing which is taped along only three sides so that pressurized air in the chest cavity can escape through an open fourth side. However, the conditions in which such wounds are typically first treated make the successful application of this type of dressing difficult to achieve in practice.

Chest seal dressings including one-way valves to promote escape of fluids, including air, from a punctured chest are disclosed in, for example, Kauth et al. U.S. Pat. No. 7,429,687.

In battlefield conditions such as may be found in the Middle East, sand carried by the wind of sandstorms, or generated by helicopter rotor wash or raised by machinery movements, may contaminate a one-way valve in known chest seal dressings, preventing complete valve closure and allowing air or sand to enter the chest cavity.

Some previously known chest seal bandages are of transparent material. In battlefield conditions at night correct placement of such chest seal bandages is difficult without use of illumination by visible light which may attract enemy attention. While they are not transparent, other medical articles such as splints are also difficult to use most advantageously without illumination by visible light.

It is somewhat awkward to place currently used chest seal bandages into a preferred location to treat an open chest wound, although some such bandages do include a handle in the form of an ear at an end or corner of such a dressing.

There is a need, therefore, for an improved medical dressing particularly for an open chest wound, which overcomes or at least mitigates the problems of the prior art arrangements.

In particular, there is a need for an improved medical dressing having a one-way valve for occluding an open chest wound while permitting pressurized air and/or other fluids to pass out of the chest cavity, and which can be made fully occlusive and able to fully exclude airborne contaminants such as sand, and there is also a need for such a dressing , as well as other medical appliances, that can be used efficiently in low-light conditions.

### SUMMARY OF THE INVENTION

A chest seal dressing as disclosed herein provides answers to the afore-mentioned needs.

In accordance with a first aspect of the invention, there is provided a medical dressing in the form of a chest seal bandage, the dressing including a one-way valve assembly that can be mounted over a wound in a patient, the valve assembly having a valve body defining a passage through the body, the valve assembly further comprising a valve member configured to permit air and/or other fluids to flow through the passage in an outward direction of the valve so that, in use, air and/or other fluids are able to exit the wound through the passage but are prevented from entering the wound through the passage; and the dressing also comprises a closure member for selectively covering the one way valve assembly, occluding it and preventing entry of sand or other airborne contaminants into the valve and thus preventing such contaminants from reaching the wound.

In one embodiment the dressing includes a flexible outer panel or sheet of material having an inner surface and an outer surface and defining an opening therethrough, the panel having an adhesive on the inner surface for attaching the panel to the skin of the patient, and the one-way valve is carried on the flexible panel, aligned with the opening through the panel.

When the closure member mentioned above is in a functioning position, the passage through the valve body is sealed, and the dressing acts as an occlusive dressing which prevents air from entering or leaving the patient's chest cavity through the wound. The closure member protects both the valve assembly and the wound against airborne contaminants such as sand and dust.

In a medical dressing or chest seal bandage in accordance with the first aspect of the invention, the closure member can be removed to allow operation of the one-way valve assembly. The passage can thus be opened up to enable pressurized air and/or other fluids to exit from the wound through the passage.

As another aspect of the invention the chest seal medical wound dressing disclosed herein may include a handling tab at each of a pair of opposite ends of the flexible panel or sheet of material, to improve the convenience of holding the dressing while placing it into a required position on a patient.

As another aspect of the invention such a medical wound dressing may include an easily identifiable marking on one of the handling tabs to improve visibility of the dressing in conditions of low levels of ambient light.

As another aspect of the invention a pattern of markings may be provided over a large area of a flexible panel of a medical wound dressing such as a chest seal bandage to facilitate seeing the dressing while applying it to a patient in conditions of low levels of ambient light.

As another aspect of the invention an area of a material highly reflective of infrared light may be provided on a medical wound dressing such as the chest seal bandage to enable the dressing to be seen using night vision equipment and a source of infrared illumination.

As another aspect of the invention a marking visible with night vision equipment may be provided on the one-way valve assembly.

As yet another aspect of the invention an area of material highly reflective of light may be provided on a medical wound dressing such as a chest seal bandage, covered by a layer of material blocking transmission of visible light yet allowing passage of infrared light, so that the highly reflective material is clearly visible using night vision goggles and infrared illumination, but is invisible without such infrared illumination.

As yet a further aspect of the invention an area of material that is luminescent in a selected range of light wavelengths in response to being exposed to light energy of a required wavelength may be provided on a medical wound dressing to facilitate visibility of the dressing at night.

As another aspect of the invention such markings that are visible using night vision equipment and a light source in a related frequency range may be provided on other medical appliances and equipment to facilitate their safe use in combat situations during darkness. For example such night vision markings can be provided on splints, tourniquets, slings, gloves, gauze, tape, IV equipment, and cricothyrotomy utensils.

The foregoing and other features of the invention will be more readily understood upon consideration of the following detailed description of the invention taken in conjunction with the accompanying drawings.

### BRIEF DESCRIPTION OF THE SEVERAL DRAWINGS

FIG. 1 is an isometric view of a chest seal bandage that is one embodiment of the invention disclosed herein.

FIG. 2 is an exploded isometric view of the chest seal bandage shown in FIG. 1.

FIG. 3A is a sectional view of a valve incorporated in the bandage shown in FIGS. 1 and 2.

FIG. 3B is a view at an enlarged scale of a detail of FIG. 3A.

FIGS. 4A, 4B, 4C, and 4D are isometric views of the valve base portion of the chest seal bandage shown in FIGS. 1 and 2.

FIGS. 5A, 5B, 5C, 5D, and 5E are views of the valve top member portion of the chest seal bandage shown in FIGS. 1 and 2.

FIG. 6 is an isometric view of the flexible valve closure member or diaphragm of the chest seal bandage shown in FIGS. 1 and 2.

FIGS. 7A, 7B, 7C, 7D, and 7E are views of a dome-shaped cover portion of the chest seal bandage shown in FIGS. 1 and 2.

FIGS. 8A, 8B, 8C, 8D, 8E, and 8F are views of a chest seal bandage that is an alternative embodiment of the chest seal bandage shown in FIGS. 1 and 2, and that includes a dome-shaped cover that can be twisted to open or occlude a valve.

FIG. 9A is a top plan view of a chest seal bandage similar to a portion of the chest seal bandage shown in FIG. 1 and FIG. 2, but without a one-way valve assembly.

FIG. 9B is an exploded view of the chest seal bandage shown in FIG. 9A.

FIG. 10A is an isometric view in humanly visible light of a bandage similar to that shown in FIG. 9A, showing indicia thereon to provide enhanced visibility at night.

FIG. 10B is a view similar to FIG. 10A showing the bandage as it would appear illuminated by near infrared light and viewed using appropriate night vision equipment.

FIG. 10C is a sectional view at an enlarged scale taken along line 10C-10C of FIG. 10A.

FIG. 10D is an isometric view of a bandage similar to that shown in FIG. 10A, but with a different arrangement of night vision indicia.

FIG. 11A is an isometric view of a one-way valve assembly and a dome-like cover attached to the valve assembly by a flexible member.

FIG. 11B is an isometric view of the valve assembly shown in FIG. 11A with the dome-like cover in place and occluding the valve assembly.

FIG. 12 is a partially cutaway side elevational view of a valve assembly equipped with a cover mounted on a center post arrangement.

FIG. 12A is an exploded sectional detail view of a latching arrangement for the center post shown in FIG. 12.

FIG. 13A is an exploded isometric view of a valve assembly in which a valve top part can be attached to the valve base portion by the use of threads, and in which the valve top portion includes a hinged cover.

FIG. 13B is an isometric view of the valve shown in FIG. 13A with the cover shown in an open position.

FIG. 13C is an isometric view of a valve similar to that shown in FIGS. 13A and 13B, with an adhesively attached removable cover shown in place.

FIG. 13D is a view similar to FIG. 13C, but with the cover partially peeled back.

FIG. 14 is an isometric view of a top member including a pair of hinged covers, for use in a valve assembly such as the one shown in FIG. 13A.

FIG. 14A is an exploded, partially cutaway view of a valve assembly including the top member shown in FIG. 14.

FIG. 15 is an isometric view of an emergency orthopedic splint including night vision indicia.

FIG. 16 is an isometric view of a tourniquet including night vision indicia.

FIG. 17 is an isometric view of a device for emergency support of an injured pelvis, including night vision indicia.

FIG. 18 is a view of a surgical glove including night vision indicia.

FIG. 19 is an isometric view of a spool of tape including night vision indicia.

### DETAILED DESCRIPTION OF PREFERRED EMBODIMENTS

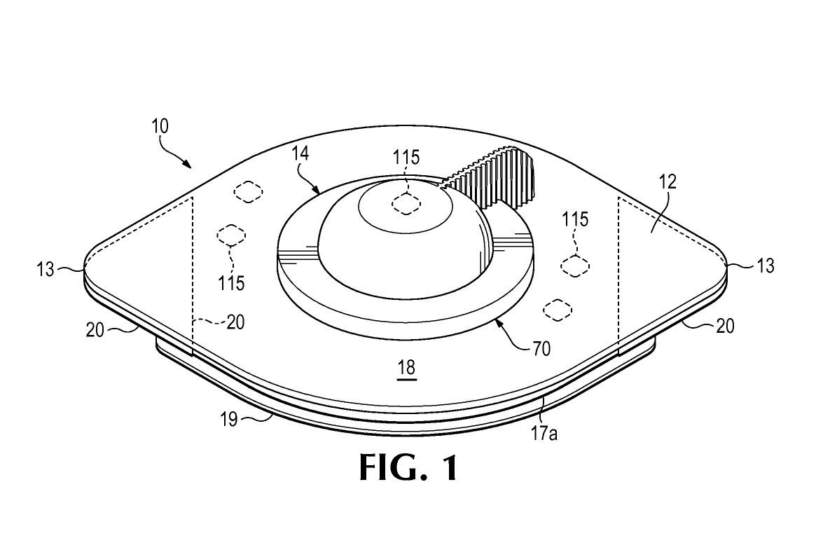

With reference to FIGS. 1 through 7E, a first embodiment of a medical dressing in the form of a chest seal bandage 10 includes a flexible base sheet, or panel, 12 and a one-way valve assembly 14 mounted on the panel. The panel 12 has an opening 12a, which is preferably located centrally of the panel, and the valve assembly 14 is mounted to the panel 12 in alignment with and covering the opening 12a.

The flexible panel 12 may be a thin sheet of flexible material of an appropriate shape. As illustrated in the present embodiment the panel 12 in the chest seal bandage 10 is generally ellipsoid in shape, with a pair of opposite ends 13 of rounded triangular shape. It will be appreciated that the actual shape of the panel is not essential to the invention. The panel 12 can be of any suitable shape for a dressing intended for a particular type of use, and could be rectangular or circular, for example.

The panel 12 can be made of any medically suitable flexible sheet material which can occlude the wound. For example, the panel 12 can be made from a film of polyurethane or polyethylene. The panel 12 is preferably made of a transparent or at least translucent material for use in a chest seal bandage, but this is not essential. As an example, the panel 12 may be of a transparent polyurethane film having a thickness of about 0.002 in (0.05 mm)

The panel 12 has an inner surface 16 intended to face toward a patient, and an outer surface 18. The inner surface 16 is covered with a layer of an adhesive 17 to enable the panel 12 to be adhered to the skin of a patient about an open chest wound. When the dressing 10 is applied, it is likely that the patient's skin in the region of the wound will be covered in blood, sweat and other contaminants such as oil and the like and may also be covered in hair. The skin surrounding the wound should ideally be cleaned prior to applying the dressing 10 but cleaning may not be totally effective, particularly if the dressing 10 is being applied in battlefield conditions. It is important, therefore, that the adhesive 17 used enables the panel to be firmly attached all the way around the wound, even where the skin is not thoroughly cleaned.

Any suitable adhesive can be used but the adhesive 17 will typically be a pressure sensitive adhesive that is transparent or translucent, such as an acrylic adhesive, in a thin layer. A relatively thick layer 17a of an additional adhesive, such as an aggressive hydrogel or a hydrocolloid gel, both of which have a good wet tack capability and so are able to adhere to damp skin and retain their tackiness over time, may also be provided, covering the layer of adhesive 17 covering the inner surface 16 of the panel 12 to a uniform thickness. Alternatively, the layer 17a of hydro gel or hydrocolloid might not extend to the outer perimeter of the panel 12, so as to leave the adhesive 17 exposed along a peripheral border portion 16a of the surface 16. This results in a panel 12 which when adhered to the skin presents a lower profile at the edges and therefore would be less likely to roll up and be detached from the skin. The border portion 16a may extend inwardly for about 5 to 10 mm, for example, along the outer perimeter of the panel 12.

The dressing 10 will usually be supplied with a non-stick, peelable, protective backing sheet 19 which covers the adhesive 17 and which can be peeled away easily when the dressing is to be applied to a person. A slender strip 19a of protective sheet material such as a thin plastic film may cover the margin of the adhesive 17 at each end of the dressing 10 and be overlapped by the ends of the peelable sheet 19, to allow a margin at each end of the peelable sheet 19 to be grasped easily for removal when the dressing is to be used.

Handling tabs 20 of a plastic or other flexible material somewhat stiffer than the panel 12 may be positioned on and attached to the inner surface 16 by the adhesive 17 at the opposite ends 13 of the panel 12 so that the dressing 10 can be grasped at the ends of the panel 12 to remove the protective backing sheet 19 and to position the dressing over the wound. The handling tabs 20 may be of a convenient shape, such as being generally triangular with a rounded outermost corner, congruent with the ends 13 of the panel 12 so as not to catch on an object and cause the dressing 10 to be dislodged, and while they need not be as flexible as the panel 12, they may advantageously be somewhat flexible, so as not to be subject to being easily snagged. The backing sheet 19 may overlap the handling tabs 20 slightly as an area to be grasped to remove the backing sheet 19 from the adhesive 17.

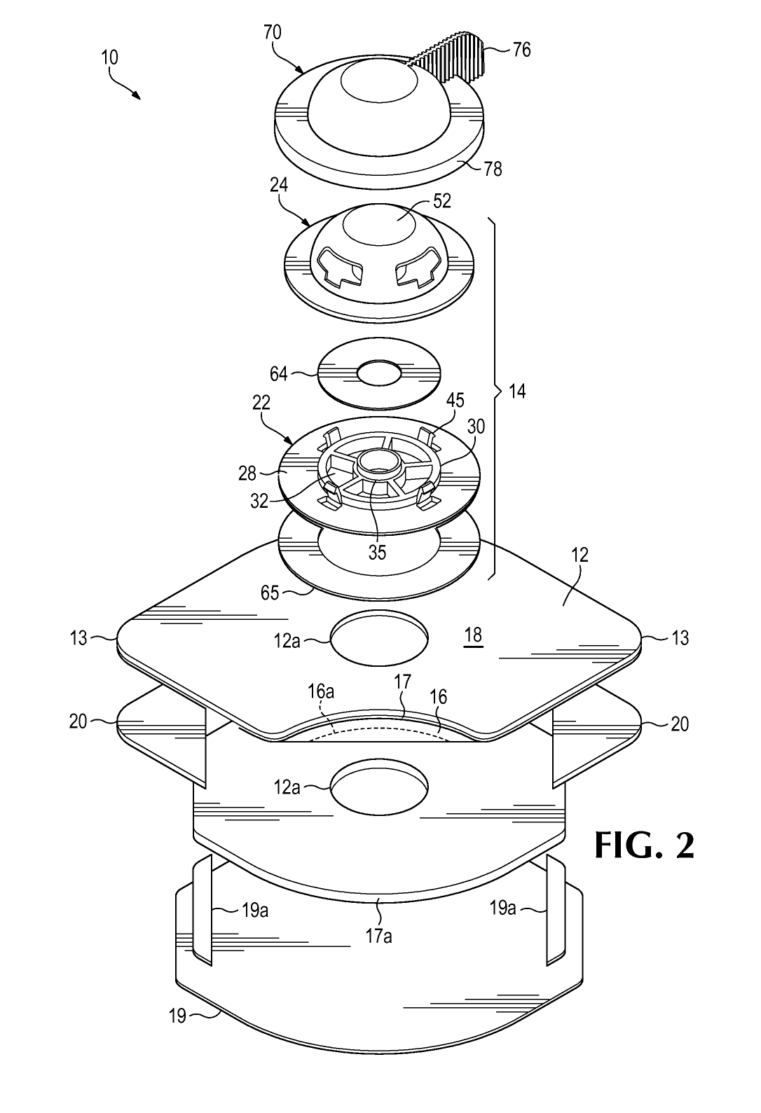

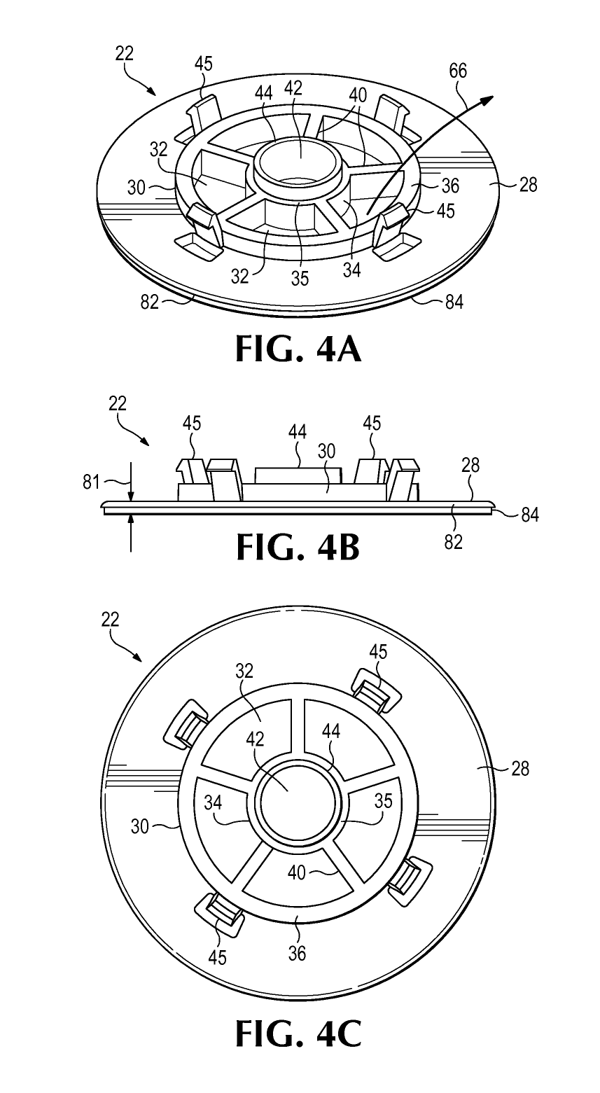

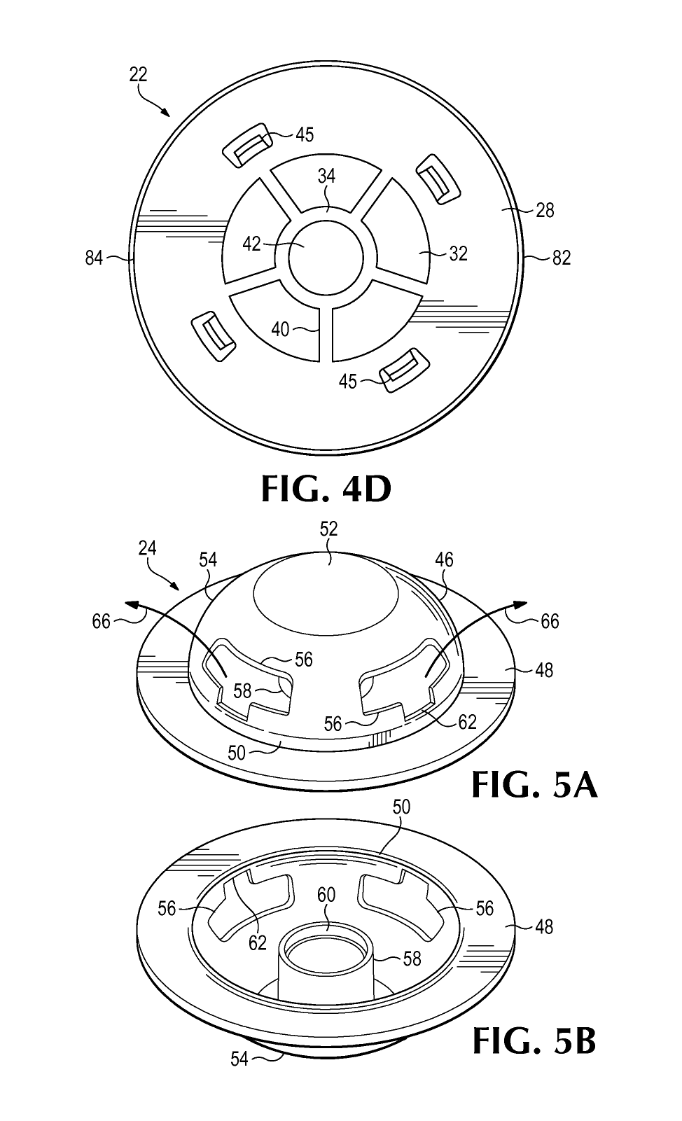

The valve assembly 14 can be seen best in FIGS. 2 through 6 and includes a valve base 22 and a valve top member 24 which together form a valve body 26. The base 22 as shown is a generally disc shaped member having a relatively thin annular flange portion 28 surrounding a raised, thicker, central portion 30. Arcuate valve openings 32 shaped as segments of an annulus extend through the central portion 30 between an inner ring 34 and a radially outer ring 36. The inner ring 34 has an annular planar outer surface 35, facing outwardly away from a wound site when the dressing 10 is in use. The arcuate valve openings 32 are arranged as a circle in the central portion 30 and are separated by radial spokes 40 which connect the inner and outer rings 34, 36, and the outer ring 36 and spokes 40 all have outer surfaces that are coplanar with the surface 35. The inner ring 34 may define a central opening 42 through the base 22, as shown in FIGS. 4A, 4C, and 4D, but the inner ring 34 could also be smaller than shown, and the central opening 42 need not be provided. A stepped annular lip 44 projects outwardly, that is to say away from the wound site in use, from a radially inner edge of the annular outer surface 35 of the inner ring 34, for reasons that will be discussed below.

Four resilient catches 45 extend outwardly from the flange 28 of the base 22 at locations spaced apart around the outer ring 36.

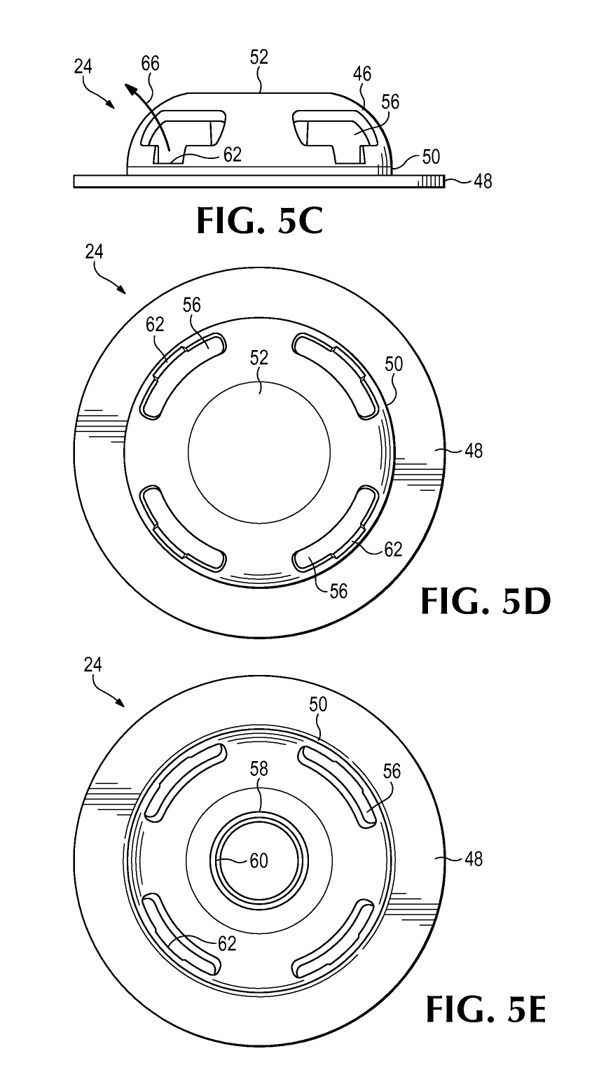

The valve top member 24 is circular in plan view, as may be seen in FIGS. 5A and 5D, having a centrally located, or inner, domed housing portion 46 and an outer annular flange portion 48 that extends radially outwardly from the base of the domed portion 46 to cover the annular flange portion 28 of the valve base 22. The domed portion 46 as shown has a low cylindrical or conical annular wall region 50 at its base, a flattened upper surface 52 and a generally curved wall portion 54 between the annular wall region 50 and the upper surface. Openings 56 are spaced circumferentially about the curved wall portion 54. The openings 56 form a fluid outlet of the valve assembly 14 as will be described in more detail later.

The valve top member 24 and base 22 are mated together. The inner diameter of the annular wall region 50 is about equal to the outer diameter of the raised central portion 30 of the valve base 22, so that the central portion 30 of the base 22 is received snugly and sealingly within the domed portion 46 of the valve top member 24 when the cover and base are assembled and held together by the catches 45. A hollow spigot 58, seen best in FIG. 5B, projects inwardly from the center of the flattened upper surface 52. An annular recess 60 may be defined by the inner surface of the free end of the spigot 58 to surround and engage with the lip 44 extending from the inner ring 34 of the valve base, and an outer end portion of the annular lip 44 can be inserted into the recess 60, optionally with an interference or snap fit to help to hold the top member 24 and the valve base 22 together. When the base 22 and the top member 24 are assembled, the flange portion 48 of the top member 24 overlies the flange portion 28 of the base 22, and the catches 45 engage seats 62 which are located in the openings 56, along the wall 50, as shown, although the seats could instead be separately located.

The valve base 22 and the valve top member 24 may both be made from a generally rigid material, such as a rigid but resilient and not brittle polymeric plastics material. Preferably, the valve base 22 and the valve top member 24 are made from a transparent or translucent polymeric material, such as polypropylene, acetyl or other suitable synthetic plastics material with a hardness defined as for polypropylene, for example, by a tensile modulus that may be in the range of 140-210 Kpsi, or 965-1448 Mpa, and the base 22 and top member 24 may be manufactured by means of injection molding, for example.

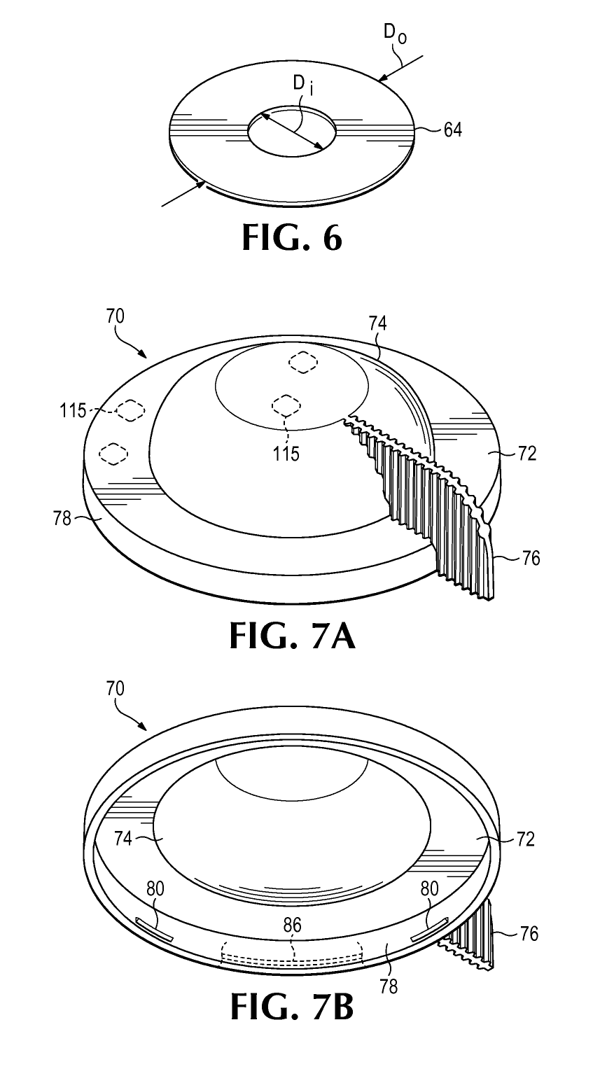

A flat, annular, flexible valve member 64 shown in FIG. 6 is fitted to the central portion 30 of the valve base 22 about the annular lip 44. The flexible valve member 64 has an outer diameter Do which may be slightly less than the outer diameter of the raised central portion 30 of the valve base 22 and has an inner diameter Di which may be slightly larger than the outer diameter of the lip 44, so that the valve member covers all of the valve openings 32 as well as at least a portion of each of the top surface 35 of the inner ring and of the upper, or outer, surface of the outer ring 36. In the present embodiment, when the top member 24 is assembled to the base 22, the annular free end of the central spigot 58 in the valve top member 24 overlies a radially inner portion of the valve member 64 to hold it in position against the annular top surface 35 of the inner ring 34 of the base. In an alternative arrangement, however, the inner diameter Di of the valve member 64 may be slightly larger than the outer diameter of the spigot 58 so that the valve member 64 is able to slide along the outer surface of the spigot 58 above the flat surface 35. The valve member 64 is made of a flexible material, such as a flexible polymer (e.g., silicone rubber) which preferably is transparent or at least translucent and which is soft enough to seat sealingly on the spokes 40 and the inner and outer rings 34, 36.

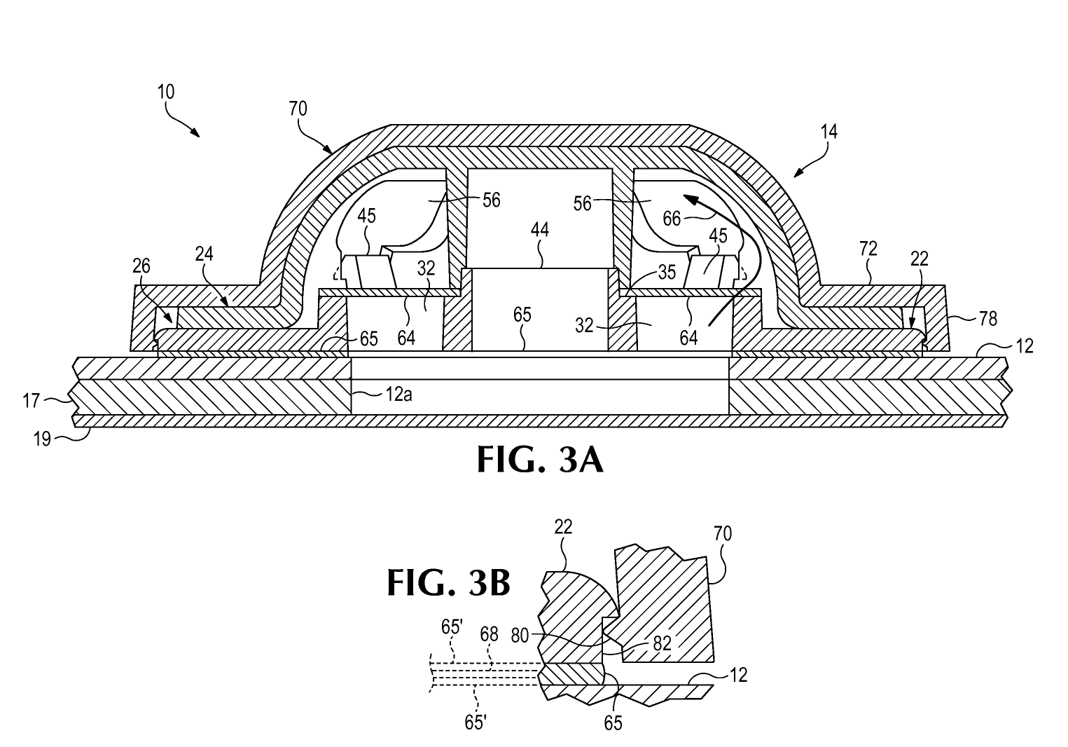

To mount the valve assembly 14 to the panel 12, a layer 65 of an adhesive material as shown in FIG. 3A, or a film 68 carrying a layer 65′ of an adhesive on each of its faces, as shown in broken line in FIG. 3B, may be used to adhere the annular flange portion 28 of the base 22 to the outer surface 18 of the panel 12, to ensure that an air-tight seal is formed between the panel 12 and the flange 28. The valve top member 24 and the valve base 22 may be held together solely by the engagement of the catches 45 with the seats 62, or they may also or instead be adhered together using a suitable adhesive material (not shown), or they may be welded together, for example by means of ultrasonic welding.

Where the base 22 and cover 24 are bonded together using adhesive or ultrasonic welding, the recess 60 and the annular lip 44 could be omitted and the inner end of the spigot 58 may have a flat surface that abuts the flat top surface 35 on the inner ring 34.

With the dressing 10 assembled as described above, the arcuate openings 32 in the base 22, the space between the base 22 and the inner surface of the valve top member 24 and surrounding the central spigot 58, and the openings 56 in the curved wall portion of the valve top member 24 together form a passage extending through the valve body 26 as indicated by the arrows 66 in FIGS. 3A, 4A, 5A, and 5C. Air and other fluids can flow outward through the passage by displacing the flexible valve member 64 temporarily, but are prevented from flowing inward by the valve member 64 when it is seated on the rings 34 and 36 and the spokes 41.

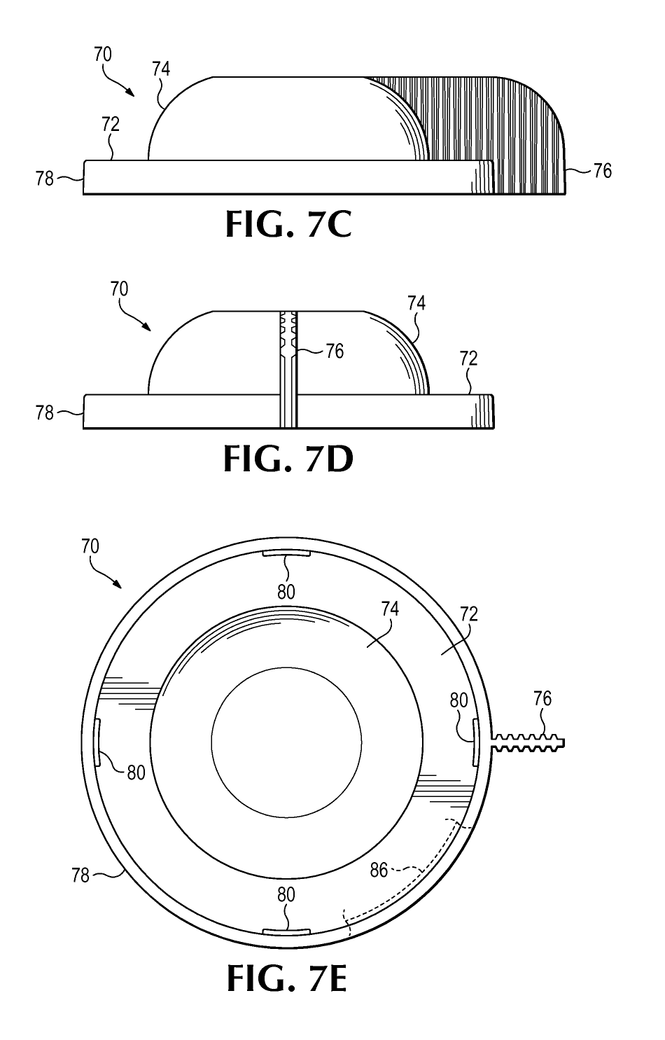

An excluder cover 70, which may be made of a stiff but somewhat resilient plastic material similar to that of the valve base 22 and valve top member 24, fits over the valve top member 24 and may be latched to the valve body 26, as shown in FIGS. 1 and 3A, to occlude the valve assembly 14 and prevent airborne matter such as dust or sand from entering into the valve assembly 14 either through the openings 56 in the valve top member 24 or between the flange 28 of the valve base 22 and the flange 48 of the valve top member 24. The cover 70 may have a radially extending generally planar annular outer portion 72 and a dome-like central portion 74 of a shape and size to conform to and fit over the valve top member 24. A handle 76 may be provided, extending radially outward from the central portion 74 and optionally having a non-slip surface configuration that may include a group of small grooves as may be seen in FIGS. 7A and 7E. An annular generally cylindrical skirt portion 78 depends from the periphery of the outer portion 72 and may include inwardly projecting catches 80 as shown in FIGS. 7B and 7E, located so as to extend radially inward into a small groove 84 beneath a narrow rim 82 extending around the upper (as seen in FIGS. 4A and 4B) edge of the valve base 22, to latch the excluder cover 70 to the valve body. The skirt portion 78 may also extend a small distance downward, or inward, beyond the depth or thickness 81 of the flange portion 28 of the valve base 22, thus pressing against the outer surface of the flexible panel 12, creating a seal against intrusion of air and airborne particles. The handle 76 may be used to aid in removing the cover 70 from the valve body when the risk of contamination by airborne materials is acceptable and it is desired to take advantage of the function of the valve assembly 14.

In another embodiment of the chest seal 10, the excluder cover 70 might be made of a softer, more flexible and elastic material such as a silicone rubber, and the catches 80 might be replaced by an inwardly projecting narrow, annular, securing rim 86, as partially shown in broken line in FIG. 7B, extending entirely around the lower edge of the skirt 78 to engage the groove 84 and elastically grip the valve body beneath the rim 82 so as to provide an airtight engagement of the excluder cover 70 with the valve base 22.

In use, the chest seal bandage or dressing 10 is attached to a patient having an open chest wound with the valve assembly 14 positioned directly over the wound and the inner surface 16 of the backing layer or panel 12 adhering to the patient's skin around the wound by virtue of the adhesive material 17 or 17a, to form an air-tight seal. With the dressing 10 in position but without the excluder cover 70 in place, air and other fluids are able to pass only out of the wound, through the passage 66, but are kept out of the wound by the valve member 64.

That is, when the pressure of the ambient air acting on an outer surface of the valve member 64 is greater than the pressure of the air and other fluids, such as blood, in the chest cavity acting on an inner surface of the valve member 64, the valve member is pressed against the outer face of the raised central portion 30 of the valve base 22 closing the arcuate openings 32 and preventing air from being drawn into the chest cavity through the valve assembly 14 and the wound. However, should the pressure of the air and/or other fluids in the chest cavity exceed the ambient air pressure, the flexible valve member 64 is at least partially lifted away from the outer surface of the central raised portion 30 of the valve base 22. This permits any pressurized air or other fluids in the chest cavity to flow outwardly through the arcuate openings 32 in the valve base, through the space between the valve base and the inner surface of the valve cover 24 surrounding the central spigot 58 and out through the openings 56 in the curved wall portion of the valve cover. The ability of pressurized air and other fluids to escape from the chest cavity through the passage 66 in the valve assembly 14 helps in treating, and in certain circumstances preventing the occurrence of, a tension pneumothorax and/or a hemothorax. When it is not desired to use the valve assembly 14 the occluding and excluding cover 70 may be placed over the valve body, protecting against contamination of the valve body 26 and the wound by airborne matter and preventing passage of fluids in either direction through the dressing 10.

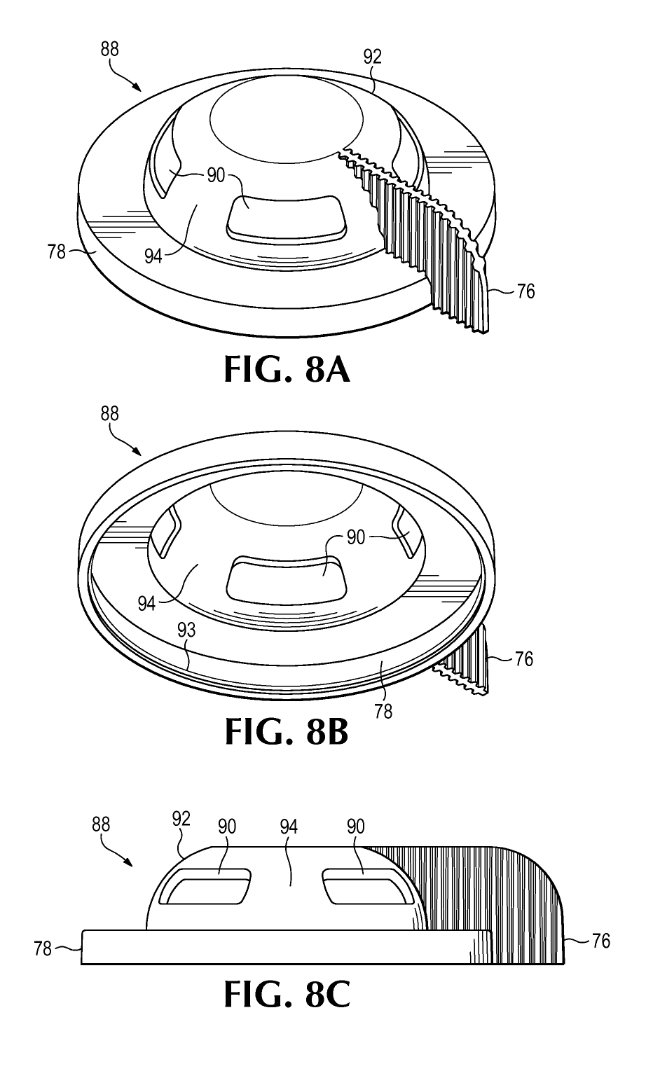

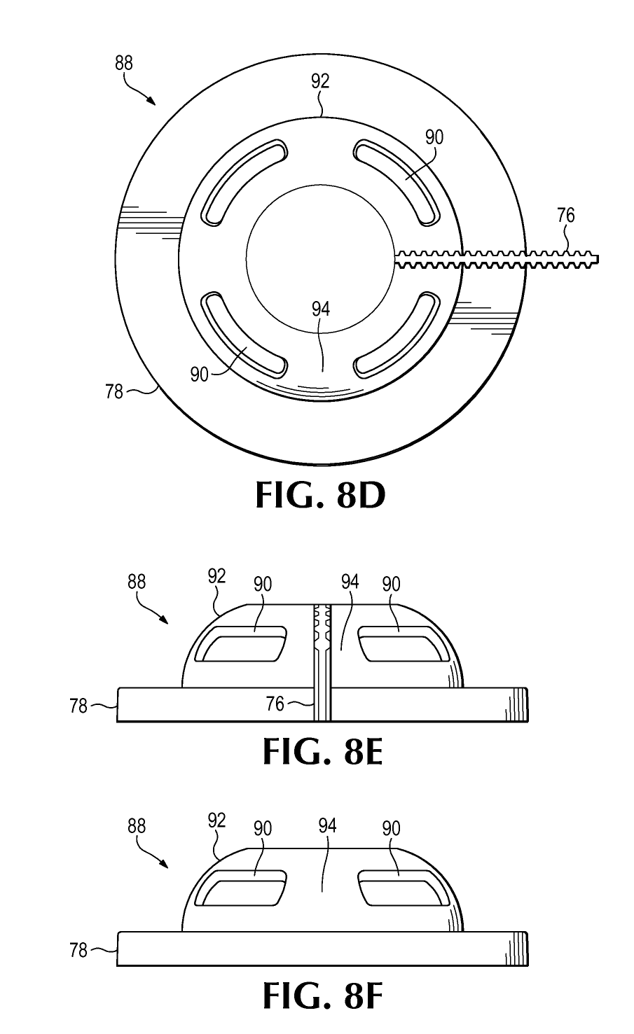

In another embodiment of the chest seal dressing, shown in FIGS. 8A-8F, an excluder cover 88 may generally be similar in shape to the excluder cover 70, but defines several openings 90 spaced about the dome-like portion 92. The openings 90 may be located so as to be aligned directly in registration with respective ones of the holes 56 in the central part 46 of the valve top member 24. The excluder cover 88 is fitted over the valve body 26 and is arranged to rotate about the valve body, such as by having a ridge 93 at its base that protrudes inwardly and fits into the groove 84 beneath the ridge 82 in the valve base 22 similar to the excluder cover 70, or by being attached via a central pin (not shown) to the top member 24 of the valve body, for example. The cover 88 could either remain on the valve body 26 or be removed as with the cover 70. Sufficient solid material 94 is provided between the openings 90 so that when the cover 88 is rotated to one position with respect to the valve top member 24 all of the holes 56 in the curved part 54 of the valve top member 24 are covered. The valve top member 24 and the excluder cover 88 must be of closely related shape and size, with the holes 56 in the valve top member 24 being small enough and spaced apart from each other far enough for all of the holes 56 to be covered by material 94 of the closed parts of the excluder cover 88, and the shape and exterior size of the valve top member 24 must fit closely against the inner surface of the excluder cover 88, so as to exclude effectively all of the airborne dust or sand that may be present and to effectively occlude the valve assembly 14 and prevent opening of the one-way valve when the excluder cover 88 is in its closed position. Detents may be provided to hold the excluder cover 88 in a selected fully open or fully occluded position of rotation about the valve body 26.

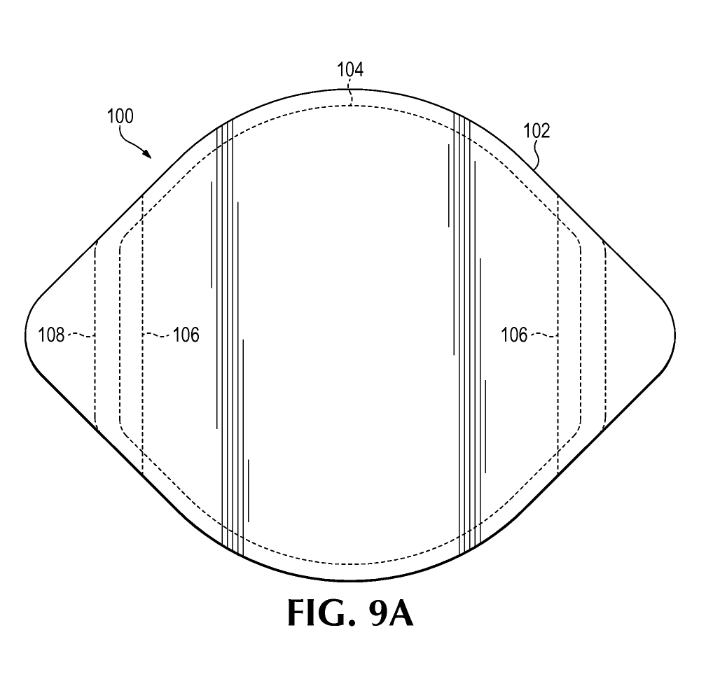

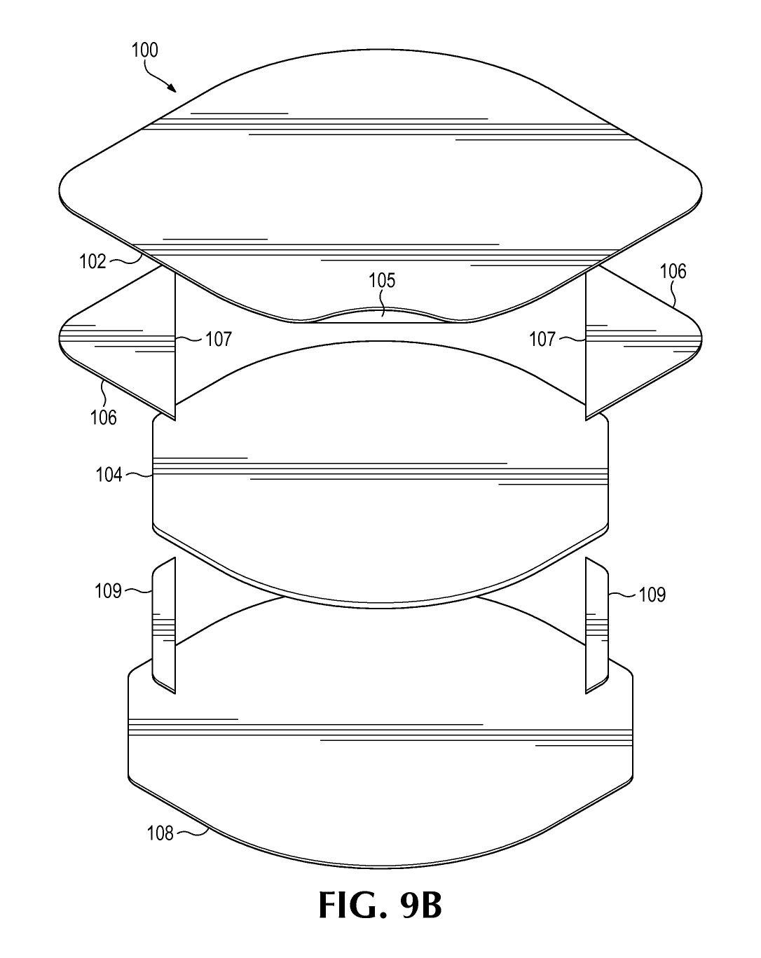

Referring next to FIGS. 9A and 9B, a bandage 100 similar in some respects to the chest seal bandage 10, but not having a valve assembly 14, is shown in some detail. A flexible main sheet or top panel 102 of sheet material, similar to the flexible panel 12 of the chest seal dressing 10, may be of flexible transparent or translucent polymeric material such as a film of polyurethane, carrying a layer 104 of an adhesive such as the gel adhesive of the layer 17 in the chest seal assembly 10. The flexible main sheet 102 might instead be of a woven textile fabric for an application other than a chest seal bandage, if an airtight seal is unnecessary. The margin of the adhesive layer 104 may be spaced inwardly apart from the periphery of the panel 102 by a small distance, and the resultant overhanging portion of the top panel 102 may have a thin layer 105 of a suitable medical adhesive such as an acrylic adhesive. At each end of the top panel 102 a handling tab 106 which may be similar to the handling tabs 20 described above, may be adhered to the material of the top panel 102, as by the layer 105 of acrylic adhesive just mentioned. As shown in FIG. 9A, the gel adhesive layer 104 may overlap an inner margin 107 of the handling tab 106. A peelable cover or backing layer 108 of PET or coated paper may be provided to protect the gel adhesive layer 104 and the acrylic adhesive layer 105 around the margin of the flexible upper panel 102 prior to use of the chest seal bandage 100. A slender strip 109 of protective sheet material such as a thin plastic film may overlie the adhesive gel 104 at each end, where the gel 104 overlaps the margin 107, leaving an area of the peelable layer 108 available to be grasped to remove the peelable layer 108 from the adhesive material of the layers 104 and 105 to facilitate removing the cover layer 108 from the adhesive gel layer 104.

### Medical Treatment During Combat in Darkness

When military personnel are operating at night it is important not to disclose their positions by use of humanly visible illumination (light in the wavelength range of about 390-750 mm, hereinafter referred to at times simply as “visible light”), and yet it is necessary to attend medically to injured personnel. While night vision equipment capable of amplifying starlight or infrared light is available, previously available chest seal bandages are usually transparent, or at least translucent, for light in the humanly visible spectrum and thus are not easily seen, even with night vision equipment, using available light such as starlight or moonlight.

Night vision goggles (NVG) such as the PVS-14 monocular in use by US military forces can do two things. One is the conversion of near infrared (nIR) light (˜750-950 nm) into visible light. Second is amplification of visible and nIR light to provide a visible image for a user.

There are two viewing modes for the NVGs. Used in a “passive” mode, the viewer at night observes amplification of only the naturally available light (starlight, moonlight, etc.). Since there is typically more visible light than nIR light under those conditions, the visible light typically overwhelms the nIR as seen by the viewer through NVGs. In an “active” mode, a source of illumination such as an LED or laser providing predominantly nIR is activated. Under dark conditions, the nIR light then overwhelms the available visible light, so most of the light available to the viewer through NVGs is in the nIR range.

Features can be included in a medical dressing such as a chest seal device to increase its visibility using NVG and thus improve usability. As mentioned previously, a chest seal dressing 10 may be transparent and thus not show up well through the NVGs. Adding a feature that is highly visible using NVG allows the medical personnel to see, for example the handling tabs 20 and edges of the dressing to increase the likelihood of successful use.

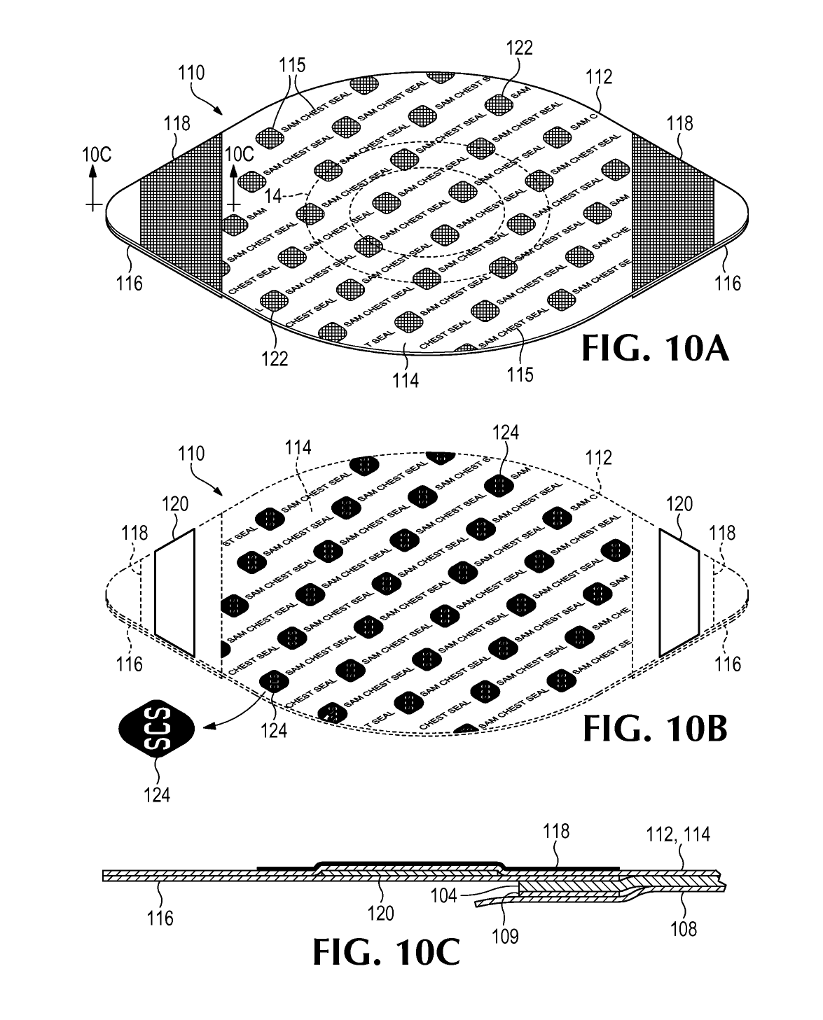

Referring next to FIGS. 10A, 10B, and 10C, in a medical dressing such as a chest seal bandage 110 that is another embodiment of the device disclosed herein a flexible backing sheet or top panel 112 is imprinted over its upper, or outer, surface 114 with night vision indicia 115 that are visible but not highly reflective in ordinary light. The NVG-visible indicia 115 may be distributed over substantially the entire area of the outer surface 114, in the form of numerous small areas of actual imprint, spread apart far enough to preserve the ability to see a patient's wound through a transparent sheet of material of the dressing 110 when sufficient light is available. Handling tabs 116 located at each of the opposite ends of the bandage 110 may also include stripes 118 visible under ordinary illumination or starlight. The stripes 118 may have a well defined size to make the handling tabs 116 easily identifiable. Making the handling tabs 116 very visible and identifiable when using NVGs is desirable to increase ease and speed of use of such a dressing. Narrower stripes 120 of reflective material also present on the handling tabs 116 are obscured by the stripes 118 in normal light, but become visible when viewed through the appropriate night vision equipment, when the bandage 110 is exposed to infrared light.

The stripes 120 may be of a reflective material or retroreflective material (such as 3M Solas 3255 or 3150, 3M Photoelectric tape 7800, 7610 or 3000x, 3M Diamond Grade tape such as 983-10). A retroreflective material typically includes tiny glass beads that direct the reflected light back in the direction of the source rather than scattering the light. This accomplishes a much higher visibility than non-retroreflective materials, and prevents bystanders or enemy combatants from seeing the reflection except within a small angle. The stripe 120 of reflective material can be laminated to the outside surface of the dressing or laminated under the dressing's top panel or backing layer 112 (between the handling tab 116 and backing layer 112). Due to the high cost of material, a narrow strip of reflective material, having, for example, a 0.5 inch width, is desired and may be used as a reflective stripe 120 on a handling tab 116.

Reflective or retroreflective material is not desired to be left uncovered since it reflects light at all frequencies, and at night sources of stray visible light (headlights, spotlights, flashlights, etc.) could cause a reflection back toward the light source and thus reveal the location of medical personnel using the chest seal dressing 10. To solve this problem, a layer of a material called “IR black,” that appears opaque and black in visible light but is tranmissive to nIR light, is put over the reflective material. This can be accomplished to provide the stripes 118 on handling tabs 116 in several ways, including by printing with an ink including the IR black material over the stripes 120, by printing the IR black in an ink over a lamination on top of the reflective material (such as the polyurethane backing layer 112 of the dressing, as shown in FIG. 10C), or by laminating an IR black tape or film onto or above the reflective or retroreflective material 120 in the chest seal dressing 110. This allows the reflection from the stripe 120 to be seen when using NVGs in the active mode but not in the passive mode. IR black inks may be specialized security inks such as Flint Group Xsys IR black, BASFs Lumogen FK 4280 or FK 4281, Paliogen Black S 084 or Perylene. Alternatively, a CMYK ink formulation consisting of C=100, M=100, Y=100, K=0 makes ink that appears black to the unaided eye but transmits nIR light. A tape or plastic film could likewise be formulated incorporating similar pigments to create the same effect. An off-the-shelf material is also available in the form of Wratten 87C photographic filter material.

Providing visible indicia in locations spread over the area of a transparent or translucent dressing allows medical personnel to visualize the shape of the dressing for accurate placement in low-light situations. A polyurethane top panel may be reflective enough to provide visible contrast with black markings on a dressing, but better visibility would be preferred. A reflective ink may be printed on the dressing, or on top of a black ink layer, to provide additional contrast. The reflective ink may be a retroreflective ink such as 3M 8000 or reflective inks such as a metallic ink (Flint Group Metalglow) or Opaque Titanium White that is highly reflective to nIR light. An ink may also be used that is formulated with pigments that absorb visible light but reflect nIR light, such as materials from Shepherd Color or Plasticolors. If an ink is used that is reflective to all frequencies, for the same reasons as above it is desired to overcoat the reflective ink with an “IR black” ink.

To permit use of a light (such as mid-IR) that is outside of the detection range of the NVGs, photoluminescent inks or pigments may be used to create a glowing effect rather than a detectable nIR reflection. The photoluminescent material would absorb in an appropriate light source's frequency and be excited to emit at a frequency detectable by the NVGs. This kind of material is available commercially from Evident Technologies (quantum dot technology), Stardust Materials, or in the form of a number of organic pigments that have that characteristic. If a photoluminescent material is to be excited by light having a frequency that is detectable by the NVGs a filter should be used with the NVGs to block the part of the light source's radiation spectrum that could be detected by the NVGs, so that the light emitted by the photoluminescent material is not overwhelmed by light from the source of illumination.

In particular, referring to FIG. 10A, the indicia 115 are visible as lettering and small black shapes 122 in daylight illumination and seen with the naked eye. In order to improve nighttime visibility of a chest seal bandage as disclosed herein, however, each of the shapes 122 may have beneath it a small imprint 124 in reflective ink, such as for example, the letters “SCS” as shown in FIG. 10B, printed in one of the reflective or photoluminescent inks mentioned above. The imprints 124 are visible when the chest seal bandage 110 is viewed using night vision goggles and illuminated by infrared or near infrared light. If the reflective ink used for the imprint 124 of the initials “SCS” is also reflective of light in the normally visible spectrum as well as infrared light, “IR black” ink may be printed over the imprint 124, covering it to provide a black shape as seen at 122 in FIG. 10A. This black shape, covering the reflective imprint 124 as seen in visible light, reduces the likelihood that the bandage might be seen at night by enemy personnel as a result of reflection of stray visible light from the imprints 124 on the medical dressing 110. Alternatively, particularly if the material of the outer layer or top panel 112 is somewhat reflective, a bottom layer of opaque black ink with open areas such as the letters “SCS” may be provided in the shape 122 as shown in FIG. 10B. The bottom layer of ink may be covered with a layer of IR black to have the entire shape 122 appear black in daylight as seen in FIG. 10A, but reflect the “SCS” under near IR illumination and seen using NVGs.

A medical dressing or bandage 110′, shown in FIG. 10D as being of the same overall shape as the dressing 110, is provided with night vision indicia including a stripe 125 of reflective or photoluminescent material, visible with night vision equipment, extending along the outer margin of the dressing or bandage 110′. A masking stripe 126 of IR black material at least as wide and preferably wider than the reflective stripe 125 covers the stripe 125 and appears to be black under humanly visible light, as shown at 127. Similar night vision indicia can be provided on bandages 110′ of other shapes and of various sizes.

It will be understood that the dressing 110, although shown with no valve assembly, could also be equipped with a valve assembly such as the valve assembly 14 described previously and shown in broken line in FIG. 10A, to make the dressing 110 particularly useful as a chest seal dressing for use during darkness. Night vision indicia 115 may also be imprinted on any of the valve assemblies disclosed herein, such as is shown in broken line on the valve assemblies 14 and 70 in FIGS. 1 and 7A.

### Alternative Valve Assemblies

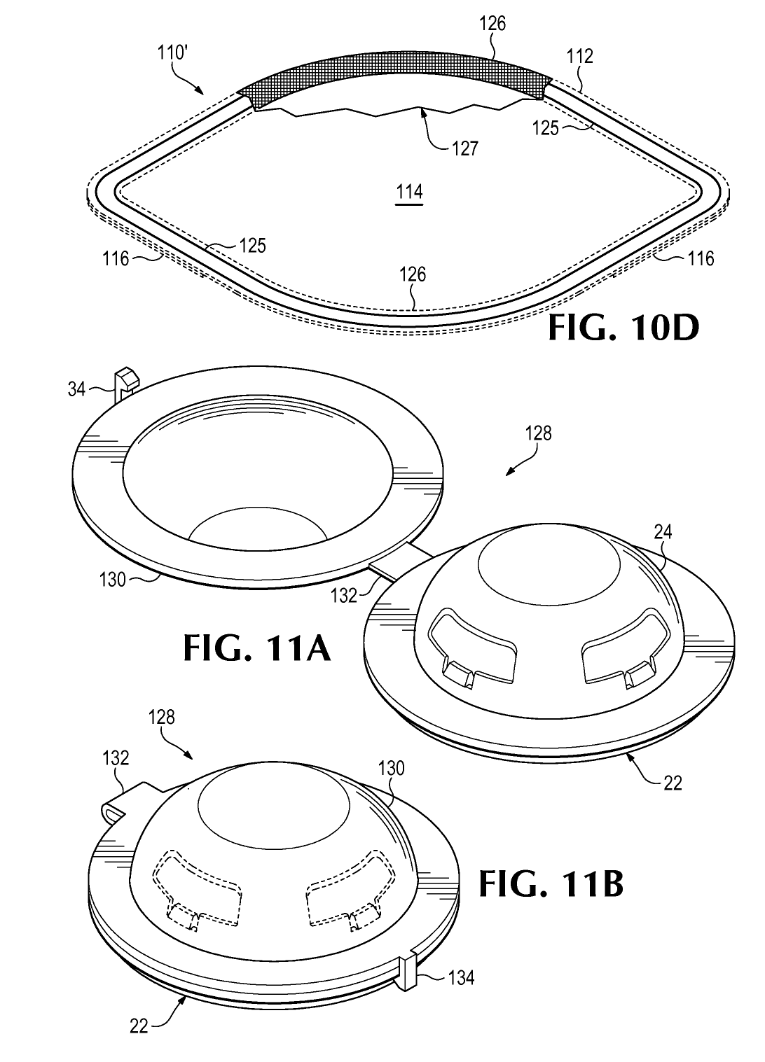

Referring now to FIGS. 11A and 11B, in a valve assembly 128, generally similar to the valve assembly 14, an excluder cover 130 may be attached to the base portion 22 of the valve assembly 128 by a so-called “living” hinge 132 formed as a relatively thin flexible part of the plastic of the valve assembly 128 during manufacture, and the excluder cover 130 may thus be of the same sort of material as which the valve base 22 is made. A catch 134 may be provided to engage the valve base 22 to keep the excluder cover 130 in a closed position as shown in FIG. 11B, to fully occlude the valve and exclude sand or other airborne contamination from the valve assembly 128.

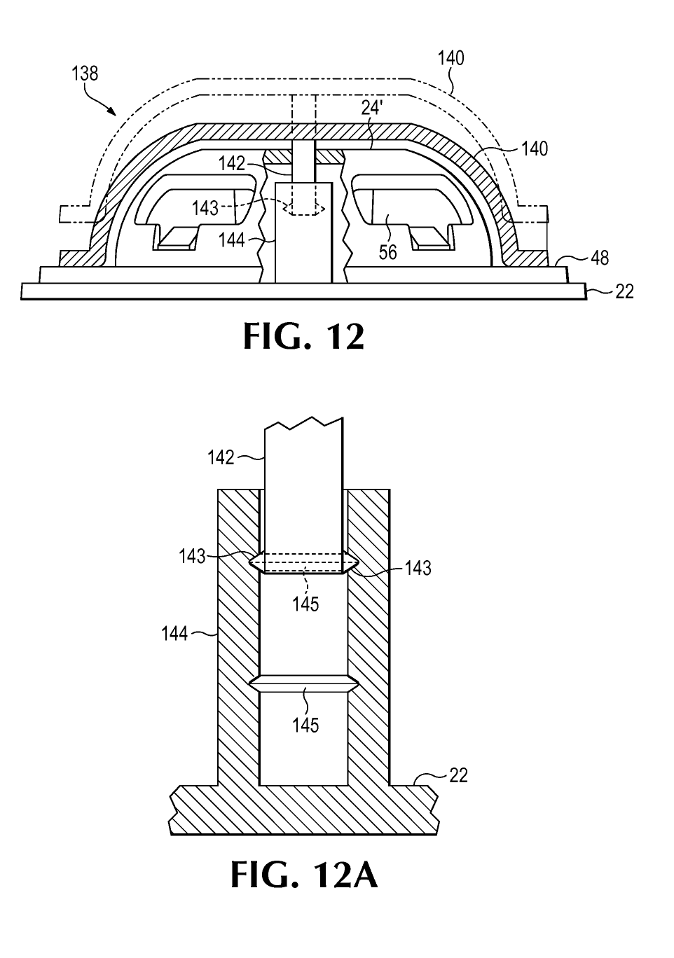

As a further alternative construction, a valve assembly 138 shown in FIG. 12 may include a cover 140 secured to the valve assembly by a movable central post 142 engaged in a tube 144 defined centrally within the valve top member 24′. A suitable resilient catch 143 (shown in FIG. 12A) may be provided to operate between the post 142 and the tube 144 as in drinking water bottle closures, engaging one of a pair of grooves 145 so as to retain the excluder cover 140 in a tightly closed condition against the flange portion 48 of the valve top member 24′ when it is desired to occlude the openings 56 in the valve assembly to exclude airborne materials. When it is desired to permit the valve assembly 138 to function as a one-way valve during use of a chest seal bandage including the valve, the excluder cover 140 may be raised to the position shown in broken line in FIG. 12 to provide space around its periphery for exhaust of air, blood, etc., that has exited from a wound and passed through the valve assembly.

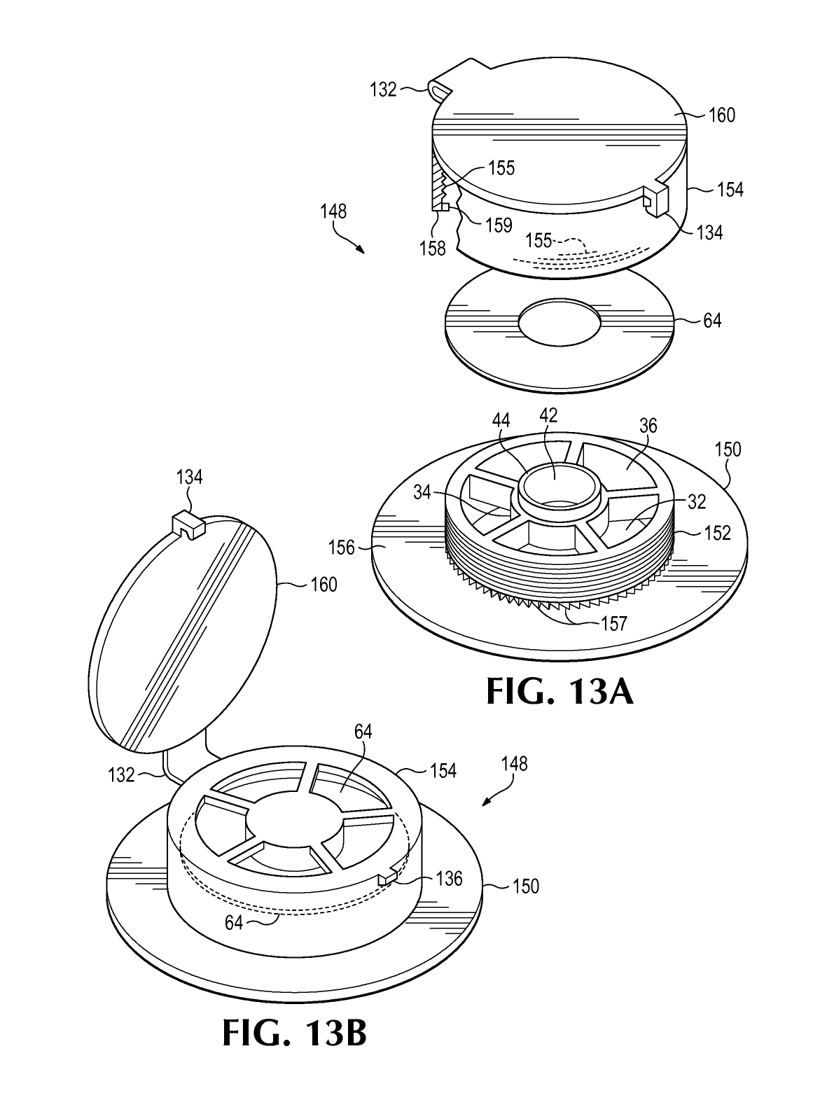

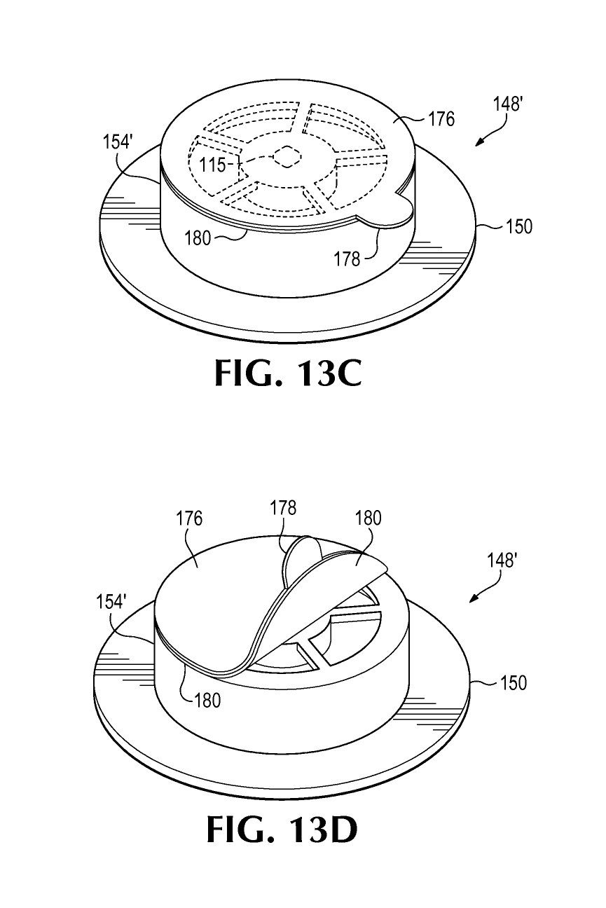

As an alternative to the use of the catches 45 and seats 62 to interconnect the base and the top member of the valve assembly 14 described above, a valve assembly 148 shown in FIG. 13A may include valve base 150, provided with helical threads 152 on an exterior portion corresponding with the center part 30 of the valve base 22 shown in FIG. 2, and a valve top member 154 may be provided with mating internal threads 155 to attach the valve top member 154 to the valve base 150 securely and with little likelihood of intrusion of sand or dust between the flange portion 156 of the valve base 150 and the bottom edge 158 of the valve top member 154. The threads 152 and 155 may be accompanied by a ratchet structure similar to those use in a child-proof medicine bottle, with teeth 157 beneath the threads 152 on the valve base 150 and one or more flexible plastic pawls 159, inside the valve top member 154, near its lower edge, so as to prevent medical personnel from disassembling the valve assembly 148. The valve assembly 148 incorporating the valve base 150 and the valve top member 154 may also include a simple flat excluder cover 160 similar in function to the excluder cover 130 shown in FIGS. 11A and 11B, attached by a living hinge 132 and including a catch 134 arranged to engage a projection 136 on the valve top member 154 or another excluder cover such as one similar to the cover 70 described above.

A valve assembly 148′ shown in FIGS. 13C and 13D is similar to the valve assembly 148 shown in FIGS. 13A and 13B, but its valve top member 154′ lacks the excluder cover 60 and the living hinge 132 shown in FIG. 13B. Instead, an excluder cover 176 may be provided in the form of a thin, preferably flexible, membrane that may be of a material such as polyethylene, polyester, polyurethane, or another plastic capable of being produced in the form of a thin film. For example, the excluder cover 176 might have a thickness in the range of 0.002-0.005 inch (0.50-1.27 mm) The excluder cover 176 is generally of a size and shape to cover and conform to the shape of the valve top member 154′, and also includes a pull-tab 178 which may be of the same film material, projecting outward beyond the valve top member 154′ in a convenient location to be grasped. The excluder cover 176 is attached to the valve body top member 154 by a layer 180 of an adhesive. The layer 180 of adhesive may be re-adherable and may have an appropriate thickness determined by the nature of the adhesive material itself, which may be an acrylic adhesive, a rubber-based adhesive, or another adhesive that has satisfactory properties for the material of the excluder cover 176 and the valve body top member 154′. The valve assembly 148′ could be provided with the excluder cover 176 attached to and closing the openings in the top member 154′, as shown in FIG. 13C. With the excluder cover 176 having an appropriate thickness it may be removed as necessary by grasping the pull-tab 178 and peeling it back as shown in FIG. 13D. If desired, night vision indicia 115, as shown in FIG. 13C, may be included on the excluder cover 176 to make it more conveniently useful during nighttime military operations.

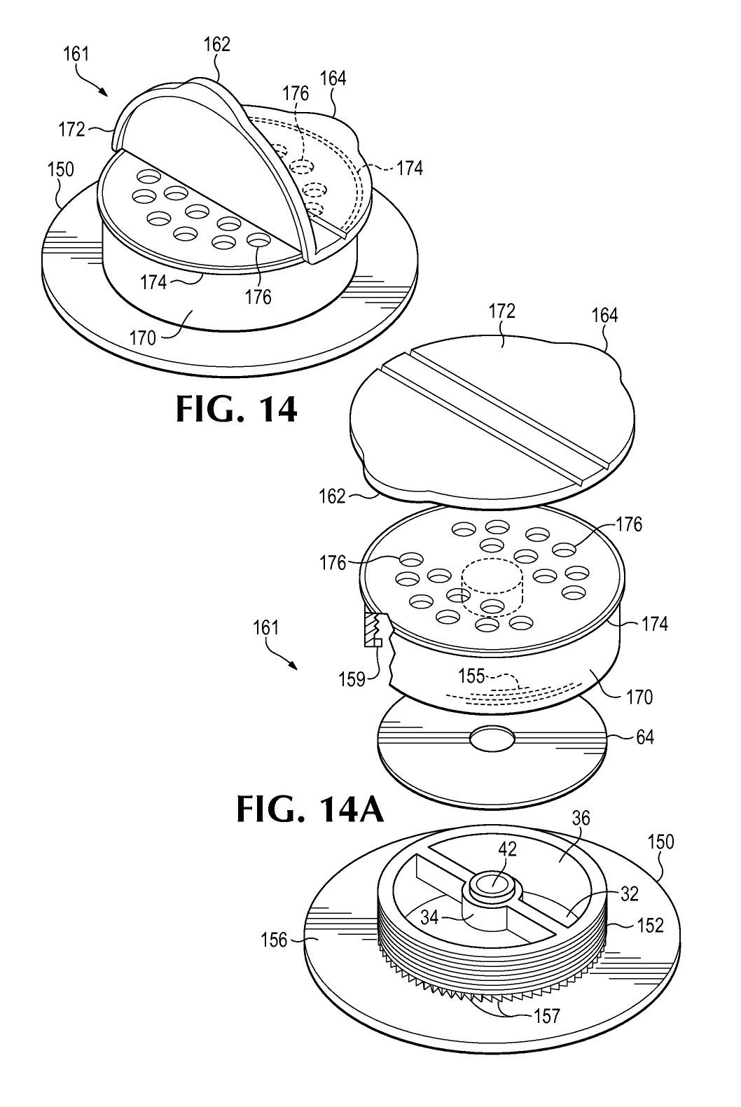

As a further alternative construction for an excluder cover in a valve assembly 161 for a chest seal dressing, as shown in FIGS. 14 and 14A, a pair of flapper lids 162 and 164 may be attached by living hinges 166 and 168 to a valve top member 170. The flapper lids 162 and 164 may be closed to bring a depending rim 172 of each lid into latching engagement with a radially protruding lip 174 on the valve top member 170 to occlude the valve assembly when desired. With the flapper lids 162, 164 open, openings 176 through the top member 170 provide a suitable passage for expulsion of air or blood that has passed through a one-way valve assembly 161 including the valve top member 170. The valve assembly 161 may be similar, otherwise to the valve assembly 148 shown in FIGS. 13A and 13B.

### Additional Medical Applications of Night Vision Improvements for Use in Combat

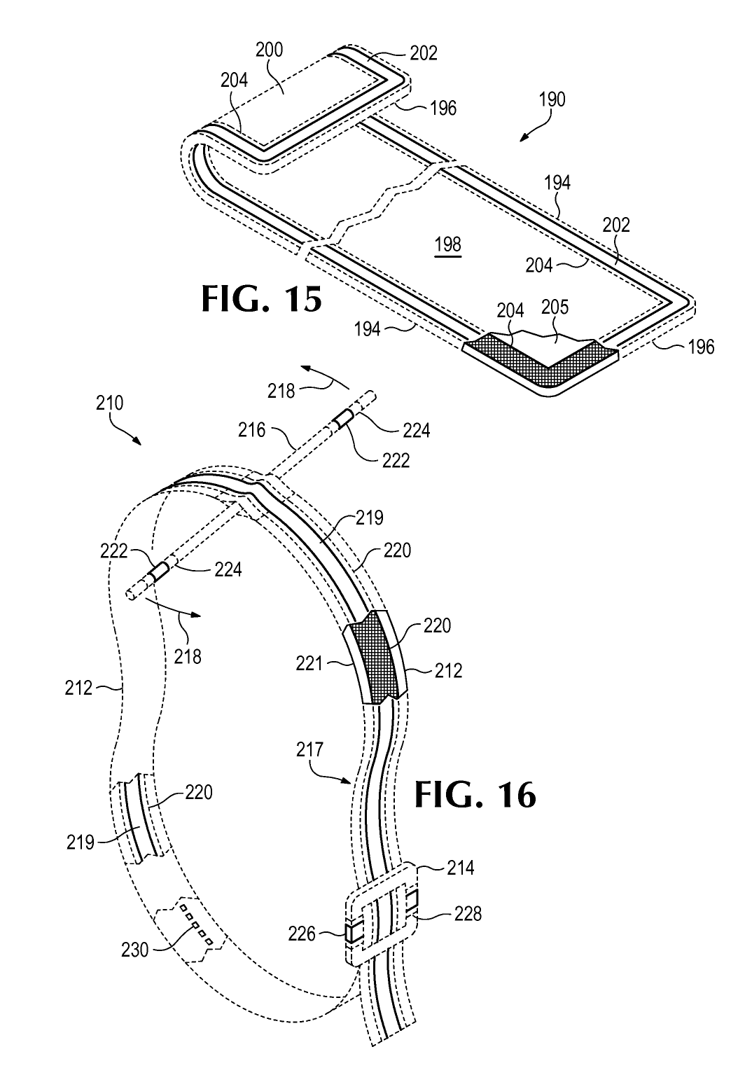

Referring now to FIG. 15, an emergency orthopedic splint 190, shown as it might be seen through night vision equipment when illuminated by near infrared light (nIR), may be of a type similar to, for example, the splints described in Scheinberg U.S. Pat. No. 4,676,233. Such splints have a soft sheet metal central member covered by a layer of closed-cell foam padding, so that the splint can be transported in a rolled or folded configuration and bent to a desired shape by a medical care provider at the time of use to immobilize a patient's injured limb. Such emergency splints may have a basically simple form including a pair of opposite faces 198 and 200 that may be flat before the splint is manipulated to place it into a desired form for a particular use. To improve the ease of use of such an emergency splint in nighttime combat conditions where night vision equipment such as NVGs capable of detecting near infrared light is available, the splint 190 is provided with night vision indicia such as a border stripe 192 extending along the lateral margins 194 and end margins 196 on at least one of the opposite major faces 198 and 200 of the splint. The border stripe 192 may be provided on the splint 190 in a manner similar to that described above with respect to application of the indicia 118, 120, 122 and 124 to the bandage 110.

That is, a stripe 202 of reflective, retroreflective, or photoluminescent material is provided along the lateral and end margins 194 and 196, spaced a small distance inwardly from the extreme edge of either face 198 or 200. The stripe 202 is then covered with a masking stripe 204 of, for example, IR black at least as wide as, and preferably slightly wider than, the stripe 202. The masking stripe 204 is shown outlined by broken line in FIG. 15, and is provided to prevent humanly visible light from impinging upon and being reflected from the reflective stripe 202, which because of its high reflectivity might, if not masked, reflect stray visible light in a direction where it might attract attention of enemy combatants. Additionally, the masking stripe 204, because of its appearance as being black in color in humanly visible light, as shown at 205 in FIG. 15 can be seen with enough contrast to assist in shaping and placing the splint 190 under conditions of minimal ambient light, both without night vision equipment and when using night vision equipment without an nIR illumination. When using NVG or other night vision equipment in combination with illumination by a source of light of a frequency not visible to humans but detectable by the night vision equipment, the reflective stripe 202 will be even more clearly visible to a medical care provider, helping to facilitate and optimize use of the splint 190 in nighttime military operations.

Night vision indicia such as the stripes 202 and 204 may be provided on the splint 190 in ways similar to the provision of the stripes 118 and 120 on the handling tabs 116 of the bandage 100. That is , the reflective stripe 202 may be provided as an imprint of an ink including reflective materials such as those described above with respect to the bandage 100, and the stripe 204 may be an imprint of ink including IR black pigment combinations such as those described above with respect to the stripes 118 and the black shapes 122. Alternatively, the stripes 202 and 204 may be flexible films of polymeric plastic material including the required reflective materials and pigments, attached to the faces 198 and 200 of the splint 190 by an appropriate adhesive material.

Another medical device whose nighttime use can be facilitated by improved visibility is the tourniquet 210, drawn in FIG. 16 to represent its appearance when illuminated by nIR and viewed using night vision equipment such as NVG. The tourniquet 210 includes a main strap portion 212 and an associated buckle 214 or another arrangement that may be used for adjusting the initial circumferential length of the strap 212 to provide an initial loop size as needed for a particular patient's limb to which the tourniquet 210 is to be applied. A winding rod 216 of suitable material may be used to twist the strap 212 as indicated by the arrows 218, in order to reduce the circumference of a loop 217 of the strap 212 while it is in place around a patient's limb.

To make the tourniquet 210 more easily usable during nighttime conditions where it is desired for the patient and medical aid personnel to remain unseen, night vision indicia may be provided on the main strap 212, as for example, a reflective stripe 219 covered by an IR black masking stripe 220, outlined in broken line, since it would not be seen if illuminated only by nIR light. The stripes 218 and 220 may be imprinted in a medial position, for example, along the length of at least one side of the strap 212, and the stripe 220 would be visible in humanly visible illumination, as shown at 221.

Similarly, bands 222 of reflective material masked by wider bands 224 of IR masking material may be provided on the rod 216 in one or more convenient locations, such as near one or each end of the rod 216, as may also be seen in FIG. 16. Indicia including a reflective marking 226, masked by an IR blacking marking 228, may also be provided on the buckle 214. The reflective stripe 219, bands 222, and indicia 226 may be affixed to the tourniquet 210 in one of the ways mentioned above with respect to the bandage 100 and the splint 190. The IR black masking stripe 220, band 224, and masking marking 228 may also be provided in one of the same ways described with respect to the bandage 100 and splint 190. Alternatively, the strap 212 may include one or more threads or strips of reflective, retroreflective, or photoluminescent material covered by a masking of IR black material and sewn or woven into the material of the strap 212 as shown at 230.

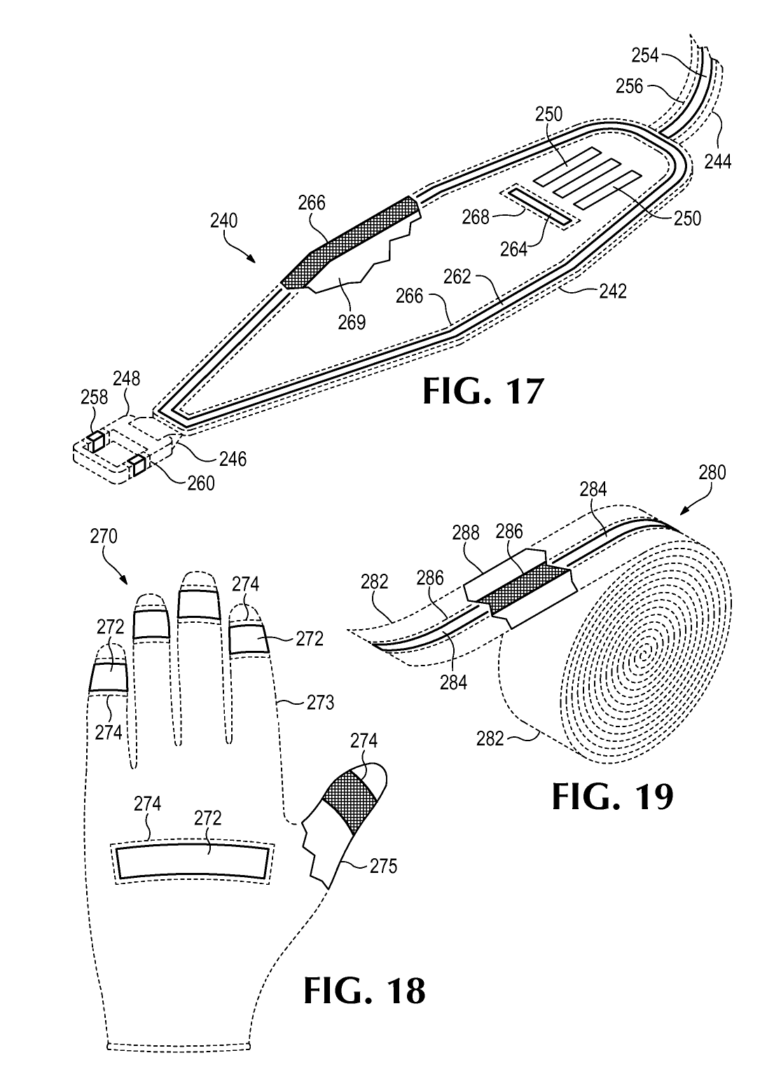

Referring next to FIG. 17, stabilizing support devices 240 known for use in immobilizing a fractured pelvis are described, for example, in U.S. Pat. Nos. 6,554,784 and 7,008,389, and in pending Polliack et al. U.S. patent application Ser. No. 12/462,754, filed Aug. 7, 2009, now U.S. Patent Application Publication No. 2011/______ A1. Such devices, often called “pelvic slings,” typically include a wide belt-like main body 242 from whose opposite ends strap members 244 and 246 extend, to be interconnected adjustably with each other by a special buckle 248. At least one of the straps 244 and 246 may be held by statistical hook-and-loop fastener material 250 to maintain a required amount of tension in the pelvic sling 240 to support a patient's injured pelvis. To facilitate proper application of such a pelvic sling 240 to a patient under nighttime conditions where personnel wish to remain unseen by enemy combatants night vision indicia may be provided on the pelvic sling 240 in a form generally similar to those previously discussed incorporating masked reflective, photoluminescent or retroreflective indicia such as a stripe 254, covered by a masking stripe 256, on each of the straps 244 and 246, a reflective, retroreflective, or photoluminescent marking 258 covered by a masking shape 260 on the buckle 248, and reflective, retroreflective, or photoluminescent indicia 262 and 264 covered by masking material 266 and 268, located on the main body 242 to disclose the shape and location of the main body 242 and the locations of the fastener material 250. As shown at 269, in humanly visible light the stripe 266 of masking material is visible as a dark stripe, hiding the reflective stripe 262.

As shown in FIG. 18, a surgical glove 270 may include night vision indicia such as reflective, retroreflective, or photoluminescent markings 272 located where they can be seen during use of the glove 270 during a medical procedure. For example the reflective indicia 272 may be provided as shown on the back of each fingertip 273, or on the back of the hand. The reflective indicia 272 are masked by masking indicia 274 visible as black shapes by an unaided human eye in daylight, as shown on the thumb at 275, or with use of night vision equipment without infrared illumination in conditions of low ambient light. The masking indicia 274 are transmissive of light in a selected spectrum such as near infrared and infrared that can be used to illuminate the gloves and enable the reflective indicia 272 to be viewed using the appropriate night vision equipment, as discussed above.

Other medically useful materials such as gauze or adhesive tape 280, which may be in the form of folded sheets or a spool or coil 282, as shown in FIG. 19, may also be provided with night vision indicia on the tape 280 in selected patterns or locations or simply as a medial stripe 284, to make it easier for the gauze or adhesive tape to be seen during use in low-light situations where it is important for involved medical or injured personnel not to be visible to personnel without special night vision equipment. The indicia 284 may be provided in the same or similar manner as those described previously, that is, by imprinting with ink including reflective, retroreflective, or photoluminescent materials, covered by a masking layer 286 of IR black material or ink containing other masking materials appropriate for a type of light not visible to an unaided human eye yet detectable using appropriate night vision equipment intended for use with the material. Thus the indicia would appear in daylight as shown at 288. Alternatively, specially prepared threads may be woven into the gauze or tape, as discussed above with respect to the tourniquet 210.

Other medically useful equipment and materials could also be provided with night vision indicia to improve their capabilities for use in nighttime combat situations. For example, indicia visible with the use of night vision equipment but not visible in darkness to the unaided human eye might be applied in the ways described above to items of intravenous injection equipment and materials, or to a cricothyrotomy tube and associated cricothyrotomy tube insertion devices, to improve their usefulness in nighttime combat situations.

While the devices mentioned hereinabove have been described specifically in connection with use of near infrared illumination and detection by night vision equipment capable of detecting near infrared and converting it to an image visible to the user of such night vision equipment, the same advantages may also be available using illumination by light in other frequency bands that are not visible to an unaided human eye, but which can be detected by appropriate equipment capable of converting a detected reflection of light in such other frequency bands to a visible image.

The terms and expressions which have been employed in the foregoing specification are used therein as terms of description and not of limitation, and there is no intention in the use of such terms and expressions of excluding equivalents of the features shown and described or portions thereof, it being recognized that the scope of the invention is defined and limited only by the claims which follow.

What is claimed is:

1. A medical chest seal dressing including a one-way valve and means for optionally and reversibly occluding the valve so as to protect against intrusion of contaminating materials into the valve from outside the dressing.

2. A medical dressing comprising:
(a) a flexible panel having an inner side and an outer side and having a layer of an adhesive material on the inner side:
(b) a one-way valve assembly mounted to the flexible panel, the valve assembly including a valve body defining a passage therethrough, between the inner and outer sides of the flexible panel, and the valve assembly being arranged to allow fluids to flow through the passage only in an outward direction with respect to the valve assembly and the dressing; and
(c) a closure member selectively covering a portion of the valve body and thereby excluding foreign matter from entering into the valve body and preventing fluid flow through the passage.

3. The medical dressing of claim 2 wherein the closure member includes a resiliently engageable catch arranged to grip the valve body to keep the closure member in a position covering said portion of the valve body.

4. The medical dressing of claim 3 wherein the valve body includes an annular flange and the catch is arranged to engage the flange.

5. The medical dressing of claim 4 wherein the flange defines an undercut groove and the catch is arranged to engage the groove.

6. The medical dressing of claim 2 wherein the valve body includes a flange having an inner side, and wherein the closure member includes an annular interior lip arranged to engage the flange on said inner side and thereby fasten the closure removably to the valve body.

7. The medical dressing of claim 2 wherein the closure member is of a flexible membraneous material and is attached removably to the valve body by a re-adherable adhesive.

8. The medical dressing of claim 2 wherein the closure member has a protruding handle that includes a non-slip grip surface including a plurality of ribs and grooves.

9. The medical dressing of claim 2 wherein the closure member is of a soft flexible material elastically gripping the valve body.

10. The medical dressing of claim 2 wherein the closure member is of a rigid but resiliently flexible material.

11. The medical dressing of claim 2 wherein the closure member remains attached to the valve body during use of the medical dressing, regardless of whether the closure member is covering the portion of the valve so as to exclude foreign matter and prevent flow through the passage.

12. The medical dressing of claim 11 wherein the closure member is attached to the valve body by a living hinge.

13. The medical dressing of claim 12 also including a second closure member attached to the valve body by a second living hinge, the living hinges being located adjacent each other and between the closure members, and both the closure member and the second closure member being selectively openable to allow fluid to flow through the passage.

14. The medical dressing of claim 11 wherein the valve body includes a circular portion that includes an exit opening defining a portion of the passage, and wherein the closure member is attached to the valve body so as to be rotatable about said circular portion, between a position in which said exit opening is unobstructed by said closure member and a position in which a portion of said closure member covers and closes said exit opening.

15. The medical dressing of claim 11 wherein said closure member is carried on a stem mounted on said valve body, the stem being movable with respect to the valve body so as to carry the closure member between an upper, open, position and a lower, closed, position in which said closure member covers said portion of the valve body and excludes said foreign matter and prevents said fluid flow through the passage.

16. The medical dressing of claim 11 wherein said valve body includes a valve base and a valve top member attached to said valve base by mating helical threads on said valve base and in said valve top member.

17. The medical dressing of claim 16 wherein said valve body includes a locking ratchet resisting disassembly of said valve body.

18. A wound dressing, comprising:
(a) a flexible wound-covering sheet of material having an outer side and an inner side intended to face toward a person's skin;
(b) a layer of an adhesive material on the inner side;
(c) indicia on said wound dressing, said indicia being visible through use of night vision equipment when viewing said outer side with said wound dressing illuminated by light energy having a wavelength outside a humanly visible light spectrum; and
(d) a layer of masking material covering said indicia, said layer of masking material being opaque to humanly visible light but transmissive of said light energy having a wavelength outside said humanly visible light spectrum.

19. The wound dressing of claim 18 wherein said indicia include an area of a reflector material that is reflective of near infrared light detectable by the night vision equipment.

20. The wound dressing of claim 19 wherein said reflector material includes retroreflective material.

21. The wound dressing of claim 18 wherein said indicia are affixed to said outer side of said wound-covering sheet.

22. The wound dressing of claim 18 wherein said indicia are carried on said flexible wound-covering sheet of material.

23. The wound dressing of claim 18 wherein said indicia are distributed over substantially all of said sheet of material, thereby disclosing a shape of said dressing.

24. The wound dressing of claim 18 wherein said indicia include an area of a photoluminescent material that is excitable by light that is not humanly visible, and wherein in response to excitation said photoluminescent material emits light detectable by the night vision equipment.

25. The wound dressing of claim 24 wherein said indicia include an area of a photoluminescent material that is excitable by light having a frequency spectrum not detectable by the night vision equipment.

26. The wound dressing of claim 25 further including a handling tab attached to said flexible sheet of material wherein said photoluminescent material is present on said handling tab.

27. The wound dressing of claim 24 wherein said photoluminescent material is present on a handling tab attached to said flexible sheet of material.

28. The wound dressing of claim 18, further including a handling tab attached to said flexible sheet of material, and wherein some of said indicia are located on said handling tab.

29. The wound dressing of claim 18 in the form of a chest seal dressing including a valve assembly carried on said flexible sheet of material.

30. The wound dressing of claim 29 wherein at least a portion of said indicia are located on said valve assembly.

31. The wound dressing of claim 18 wherein said indicia include an area of a photoluminescent material that emits light that is outside a spectrum of humanly visible light, but that has a frequency detectable by the night vision equipment.

32. A medical article for use by a medical care provider in providing medical care during darkness, comprising:
(a) a main member;
(b) night vision indicia carried on said main member and visible using night vision equipment when said indicia are illuminated by light having a wavelength that is invisible to a unaided human eye; and
(c) a masking material covering said indicia and preventing humanly visible light from being transmitted from said indicia to an unaided human eye.

33. The medical article of claim 32 wherein said masking material is visible in daylight to an unaided human eye.

34. The medical article of claim 32, wherein the medical article comprises an orthopedic splint.

35. The medical article of claim 34 wherein said indicia are provided as a stripe extending along a margin of said orthopedic splint.

36. The medical article of claim 32 wherein said medical article is a tourniquet.

37. The medical article of claim 36 wherein said tourniquet includes a main strap and said indicia include a stripe extending along said main strap.

38. The medical article of claim 36 wherein said tourniquet includes a winding rod and at least some of said indicia are located on said winding rod.

39. The medical article of claim 36 wherein said tourniquet includes a buckle and at least some of said indicia are located on said buckle.

40. The medical article of claim 32 wherein said medical article is a pelvic sling.

41. The medical article of claim 40 wherein said pelvic sling includes a belt-like main body and at least a part of said indicia are located on said main body.

42. The medical article of claim 40 wherein said pelvic sling includes a strap extending from an end of a belt-like main body and wherein at least a part of said indicia are located on said strap.

43. The medical article of claim 40 wherein said pelvic sling includes a buckle interconnecting a strap with a belt-like main body and wherein at least a part of said indicia are located on said buckle.

44. The medical article of claim 32, wherein said medical article is a surgical glove, and wherein at least some of said indicia are located on a finger of said glove.

45. The medical article of claim 32 wherein said medical article comprises gauze and said indicia are incorporated into said gauze.

46. The medical article of claim 32 wherein said medical article is a tape having a length and a width and an adhesive carried on a surface thereof and wherein said indicia are located at least at intervals along said tape.

47. The medical article of claim 46 wherein said indicia extend longitudinally along said tape.

48. A wound dressing comprising:
(a) a flexible panel of sheet material having an inner side and an outer side and a pair of opposite ends;
(b) a layer of a flexible adhesive material on the inner side;
(c) a respective handling tab of flexible sheet material attached to said inner side by said adhesive at each of said opposite ends of said flexible panel; and
(d) a peelable protective backing sheet covering said adhesive material, a portion of said backing sheet overlapping one of said handling tabs, as an area of said backing sheet to be grasped to peel said backing sheet away from said adhesive material.
